(19) United States
(12) Patent Application Publication
(10) Pub. No.: US 2025/0352365 A1
(43) Pub. Date: Nov. 20, 2025

(54) SELF-EXPANDING STENTS WITH DEFORMABLE RETENTION MEMBERS

(71) Applicant: BOSTON SCIENTIFIC SCIMED, INC., Maple Grove, MN (US)

(72) Inventors: ROBB MORSE GAVALIS, Westborough, MA (US); JAMES J. SCUTTI, Norwell, MA (US)

(73) Assignee: BOSTON SCIENTIFIC SCIMED, INC., Maple Grove, MN (US)

(21) Appl. No.: 19/210,579

(22) Filed: May 16, 2025

Related U.S. Application Data
(60) Provisional application No. 63/649,178, filed on May 17, 2024.

(51) Int. Cl. A61F 2/848; A61F 2/90

(52) U.S. Cl. CPC A61F 2/848; A61F 2/90; A61F 2210/0014; A61F 2220/0016; A61F 2230/0095

(57) ABSTRACT

An implantable medical device comprising an elongated body having a first end, a second end, and a lumen extending therebetween, a saddle region defined between the first end and the second end, a retention member at the first end, the second end, or both, wherein the retention member extends substantially traverse to a longitudinal axis of the implantable medical device and is configured to deform responsive to an application of a force along a longitudinal axis of the implantable medical device.

### CROSS-REFERENCE TO RELATED APPLICATIONS

The application claims the benefit of U.S. Provisional Patent Application Ser. No. 63/649,178, filed on May 17, 2024, the disclosure of which is incorporated herein by reference.

### FIELD

The disclosure relates generally to the field of implantable medical devices that extend across anatomical structures, such as for establishing a connection and/or fluid communication between the anatomical structures. More particularly, the disclosure relates to devices, systems, and methods for establishing a connection and/or fluid communication between anatomical structures via longitudinally self-expanding stents with deformable retention members.

### BACKGROUND

Various devices such as stents are known for extending across anatomical structures for various purposes. For instance, various stents are known for establishing connections between anatomical structures. Some such connections are made simply to hold tissue in apposition, whereas some such connections also establish fluid communication between anatomical structures such as organs, cavities, lumens, passages, etc. In some instances, it is desirable to create a semi-permanent or permanent anastomosis allowing fluid flow or drainage from one anatomical structure to another anatomical structure. In general, in various procedures or uses of a stent extending across anatomical structures, such as to create an anastomosis, it may be desirable for the stent to remain in place for a prolonged period of time (e.g., days, weeks, months, even upwards of six to twelve months).

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In a first example, an implantable medical device is provided. The medical device comprising: an elongated body having a first end, a second end, and a lumen extending therebetween; a saddle region defined between the first end and the second end; a retention member at the first end, the second end, or both, wherein the retention member extends substantially traverse to a longitudinal axis of the implantable medical device and is configured to deform responsive to an application of a force along a longitudinal axis of the implantable medical device.

Alternatively or additionally to any of the examples herein, in another example, wherein the retention member is configured to elastically deform between: a first configuration in an absence of the application of the force; and a second configuration responsive to the application of the force.

Alternatively or additionally to any of the examples herein, in another example, wherein the retention member, the saddle region, and the elongated body are formed of the same material.

Alternatively or additionally to any of the examples herein, in another example, wherein the material is a shape memory material.

Alternatively or additionally to any of the examples herein, in another example, wherein the retention member is integral with the saddle region.

Alternatively or additionally to any of the examples herein, in another example, wherein the retention member includes a flared region.

Alternatively or additionally to any of the examples herein, in another example, wherein the flared region terminates in an arch.

Alternatively or additionally to any of the examples herein, in another example, wherein the flared region terminates in a rolled flange.

Alternatively or additionally to any of the examples herein, in another example, wherein the rolled flange terminates in a plurality of anti-migration features.

Alternatively or additionally to any of the examples herein, in another example, wherein the plurality of anti-migration features are a plurality of teeth, wherein the plurality of teeth are configured to contact a tissue wall when the retention member is in a first configuration and remain in contact with the tissue wall when the retention member is in a second configuration.

Alternatively or additionally to any of the examples herein, in another example, wherein the plurality of anti-migration features are uncoated, and wherein at least the saddle region is coated.

Alternatively or additionally to any of the examples herein, in another example, wherein the elongated body, the saddle region, and the retention member are formed of interwoven filaments.

Alternatively or additionally to any of the examples herein, in another example, wherein the retention member includes a first retention member at the first end and a second retention member at the second end.

Alternatively or additionally to any of the examples herein, in another example, wherein the first retention member and the second retention member are the same shape and same size.

Alternatively or additionally to any of the examples herein, in another example, wherein the retention member includes a longitudinal projection extending therefrom, and wherein the longitudinal projection is: configured to be disposed in a gap between a tissue wall and the retention member when the retention member is in a first configuration; and configured to contact the tissue wall when the retention member is in a second configuration.

In another example a self-expanding implantable medical device is provided. The device comprising: an elongated body having a first end, a second end, and a lumen extending between the first end and the second end; retention members including a first retention member at the first end and a second retention member at the second end, wherein the retention members extend substantially traverse to the longitudinal axis of the implantable medical device and are configure to undergo elastic deformation between a first configuration in an absence of an application of a force along a longitudinal axis of the implantable medical device and a second configuration responsive of the application of the force; and a longitudinally expandable saddle region defined between the first retention member and the second retention member, wherein the longitudinally expandable saddle region is configured to extend between a first tissue wall and a second tissue wall, the first retention member is configured to anchor the implantable medical device with respect to the first tissue wall, the second retention member is configured to anchor the implantable medical device with respect to the second tissue wall.

Alternatively or additionally to any of the examples herein, in another example, wherein the longitudinally expandable saddle region has: a first length along the longitudinal axis of the longitudinally expandable implantable medical device in the absence of the application of the force; a second length along the longitudinal axis responsive to the application of the force, wherein the second length is larger than the first length, and a difference between the second length and the first length is in a range from about 5 millimeters to about 2 centimeters.

In another example a self-expanding implantable stent is provided. The stent comprising: an elongated body having a first end (e.g., a proximal end), a second end (e.g., a distal end), and a lumen extending between the first end and the second end; rolled retention members including a first rolled retention member at the first end and a second rolled retention member at the second end, wherein the first and second rolled retention members extend substantially traverse to the longitudinal axis of the implantable medical device and are configure to undergo elastic deformation between a first configuration in an absence of an application of a force along a longitudinal axis of the implantable medical device and a second configuration responsive of the application of the force; a saddle region defined between the first rolled retention member and the second rolled retention member, wherein the saddle region is configured to extend between a first tissue wall and a second tissue wall; a first plurality of anti-migration features extending substantially longitudinally from an end of the first rolled retention member and being configured to anchor the implantable medical device with respect to the first tissue wall; and a second plurality of anti-migration features extending substantially longitudinally from an end of the second rolled retention member and being configured to anchor the implantable medical device with respect to the second tissue wall.

Alternatively or additionally to any of the examples herein, in another example, wherein: the rolled retention members have a substantially circular cross-sections; the first plurality of anti-migration features comprise teeth that are disposed uniformly about a substantially circular end of the first rolled retention member; and the second plurality of anti-migration features comprise teeth that are disposed uniformly about a substantially circular end of the second rolled retention member.

Alternatively or additionally to any of the examples herein, in another example, wherein: the first rolled retention member is configured to unroll in a first direction about the longitudinal axis of the implantable medical device responsive to the application of the force; and the second rolled retention member is configured to unroll in a second direction that is opposite from the first direction.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates a biliary system of a patient;

FIG. 1B illustrates the biliary system of FIG. 1A in alternative detail;

FIG. 2A illustrates a view of an implantable medical device in a first configuration in accordance with various principles of the present disclosure;

FIG. 2B illustrates a view of an implantable medical device in a second configuration in accordance with various principles of the present disclosure;

FIG. 2C illustrates a section view of the implantable medical device in accordance with various principles of the present disclosure;

FIG. 2D illustrates another section view of the implantable medical device in accordance with various principles of the present disclosure;

FIG. 2E illustrates a view of an implantable medical device in a first configuration in accordance with various principles of the present disclosure;

FIG. 2F illustrates a view of an implantable medical device in a second configuration in accordance with various principles of the present disclosure;

FIG. 2G illustrates a section view of an implantable medical device;

FIG. 3A illustrates a view of an implantable medical device in a first configuration in accordance with various principles of the present disclosure;

FIG. 3B illustrates a view of an implantable medical device in a second configuration in accordance with various principles of the present disclosure;

FIG. 4A illustrates a view of an implantable medical device in a first configuration in accordance with various principles of the present disclosure;

FIG. 4B illustrates a view of an implantable medical device in a first configuration in accordance with various principles of the present disclosure;

FIG. 5A illustrates a view of an implantable medical device in a first configuration across a schematic representation of apposed tissue walls in accordance with various principles of the present disclosure; and

FIG. 5B illustrates a view of an implantable medical device in a first configuration across a schematic representation of apposed tissue walls in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “channel” or “bore” or “lumen” or “passage” is not limited to a circular cross-section. As used herein, a “free end” of an element is a terminal end at which such element does not extend beyond. Finally, reference to “at” a location or site is intended to include tissue at and/or about the vicinity of (e.g., along, adjacent, etc.) such location or site.

In accordance with various principles of the present disclosure, implantable medical devices are formed to extend across adjacent or apposed anatomical structures. It will be appreciated that such implantable medical devices may be referenced herein as scaffolds, grafts, stents, etc., without intent to limit. In accordance with various further principles of the present disclosure, such implantable medical devices are formed to hold anatomical structures in apposition. Even more particularly, such stents may be formed to establish a flow or access passage between the apposed anatomical structures. The anatomical structures may be lumens, channels, vessels, passages, cavities, organs, cysts, pseudocysts, etc., the present disclosure not necessarily being limited to use between particular anatomical structures. For the sake of convenience, and without intent to limit, reference may be made to holding tissue walls in apposition, it being appreciated that such is only one example of anatomical structures and association therewith for which principles of the present disclosure are applicable.

An implantable medical device formed in accordance with various principles of the present disclosure includes an elongated body shiftable from a delivery configuration to a deployed configuration. In the delivery configuration, the elongated body is generally compact and/or constricted to be capable of transcatheter delivery through a patient's body without requiring an open surgical procedure. Accordingly, in the delivery configuration, the implantable medical device may be compressed and/or elongated or otherwise configured to be able to fit within a generally tubular delivery device (e.g., endoscope, catheter, shaft, etc.) capable of transluminal delivery through the patient's body. Once the implantable medical device is delivered to the desired anatomical site (which may be alternately referenced herein as a treatment site, deployment site, delivery site, etc., without intent to limit), the implantable medical device may be allowed to shift into a deployed configuration. In the deployed configuration, the implantable medical device may be in a generally expanded configuration. In the deployed configuration, the implantable medical device may define a saddle region with a first end and a second end and one or more retention members (which may alternately be referenced herein as a flange) at or along each end thereof. It will be appreciated that terms such as at or on or adjacent or along an end may be used interchangeably herein without intent to limit unless otherwise stated, and are intended to indicate a general relative spatial relation rather than a precisely limited location. The retention members are sized, shaped, configured, and/or dimensioned to retain the implantable medical device with respect to the deployment site. More particularly, the size, shape, configuration, and/or dimensions of the retention members may be selected to seat against a body wall extending radially outwardly from the body passage through which the saddle region of the implantable medical device is positioned. As such, the retention members are transverse to, and typically extend substantially perpendicular to, the saddle region of the implantable medical device. Typically, the retention members are wider (in a radial direction transverse to the longitudinal axis of the body passage) than the saddle region. It will be appreciated that reference to a body passage includes naturally-existing passages as well as medically-created passages (e.g., a passage created with the use of a medical instrument). In some embodiments, the retention members herein can be configured to undergo motion (e.g., translation and/or rotation) responsive to application of a force to the implantable medical devices herein.

In some aspects of the present disclosure, the saddle region defines a lumen therethrough to allow passage of materials (e.g., a bodily fluid such as bile) from one anatomical structure, through the lumen of the saddle region, and to another anatomical structure. The retention members of the implantable medical device retain the implantable medical device in place with respect to the two anatomical structures. Additionally or alternatively, the retention members hold in apposition the tissue of the anatomical structures between/across which the implantable medical device is positioned.

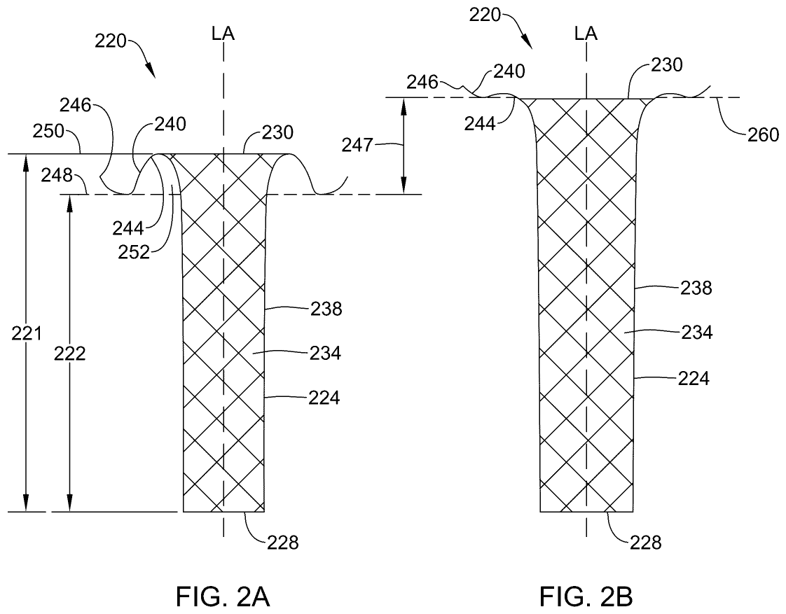

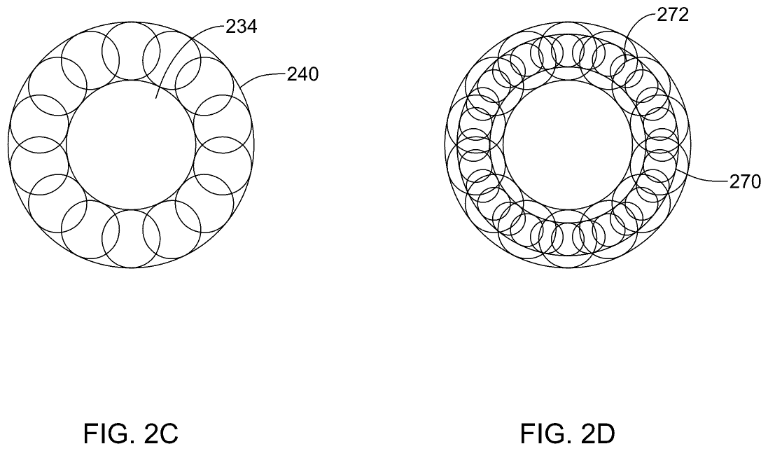

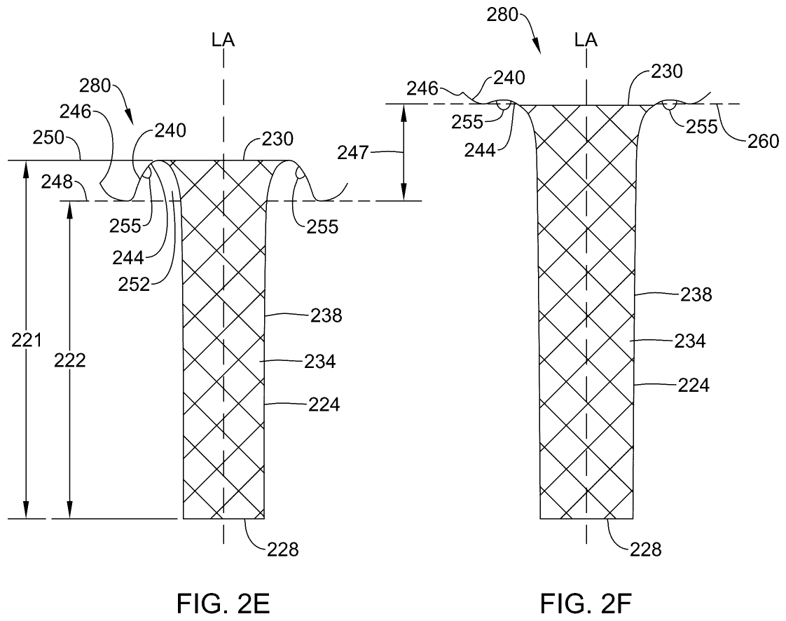

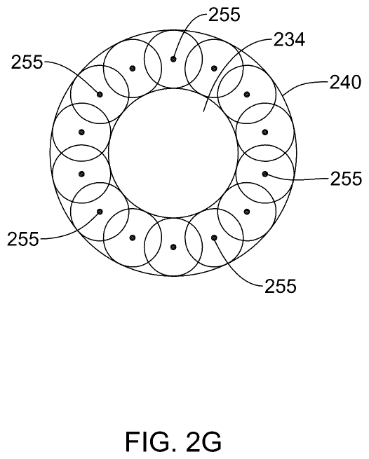

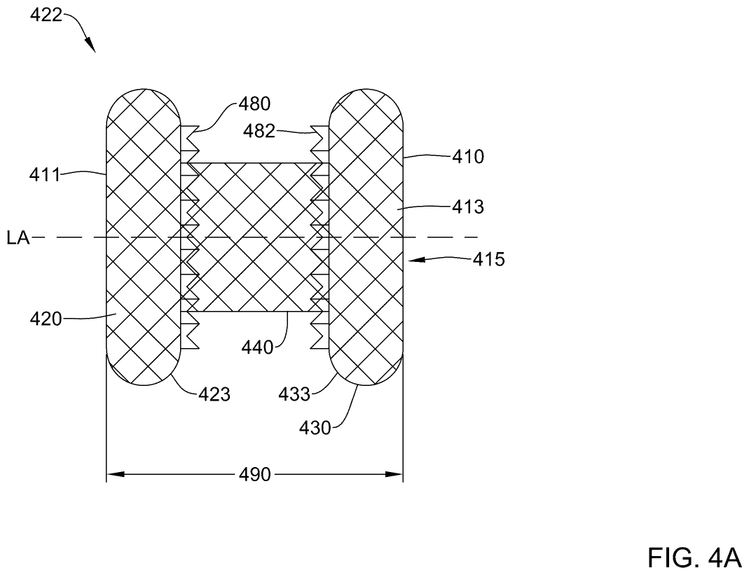

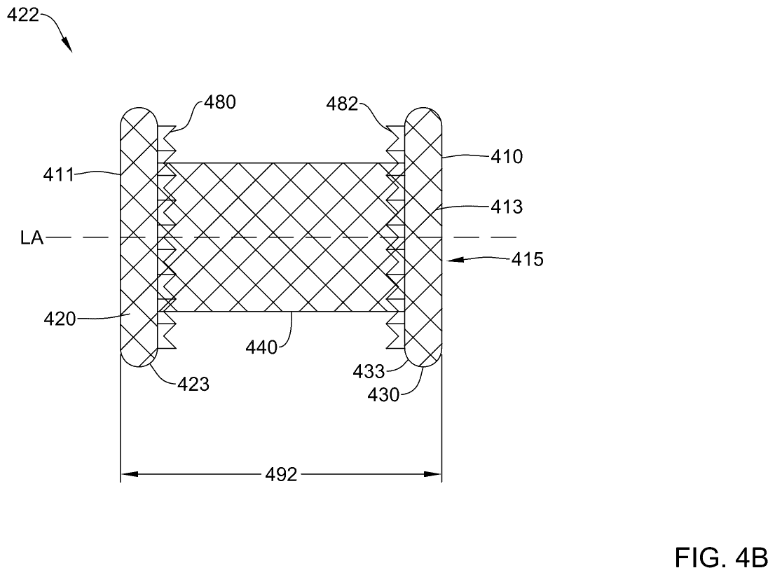

In accordance with various principles of the present disclosure, the implantable medical device, including the saddle region and retention members, is partially or fully coated with a material which prevents passage of material through the walls thereof, such as through the wall of the saddle region. Such coating typically inhibits tissue ingrowth into the implantable medical device wall. However, tissue ingrowth may be useful for inhibiting migration of the device with respect to the implant site. For instance, in some embodiments a portion of the implantable medical devices herein can remain uncoated. For example, projections (e.g., projections 255 as illustrated in FIG. 2E) and/or anti-migration features (e.g., 480, 482 as illustrated in FIG. 4A) can be uncoated to promote tissue in growth, while a remainder of the implantable medical devices can be coated.

In accordance with various principles of the present disclosure, longitudinally self-expanding implantable medical devices are provided. For instance, the longitudinally self-expanding implantable medical devices may be employed for treatment of biliary obstructions as described herein. As bodily structures shift, implantable medical devices such as stents that are coupled to the bodily structures may be prone to undergo migration.

Some previous approaches seek to mitigate movement of the stent by locking mechanisms, suturing, or otherwise employing anti-migration features that are configured to reduce any movement (e.g., longitudinal movement) of a stent. Such approaches may employ a flexible stent in combination with the locking mechanism, suturing, or otherwise employing anti-migration features that are configured to reduce any movement (e.g., longitudinal movement) of a stent. That is, such approaches may attempt to provide a degree of movement via elongation of a material of the stent itself. However, the degree of movement afforded via elongation of the material itself may be insufficient to accommodate a degree and/or reoccurrence of natural body movements. Other previous designs may generally seek to resist the natural motion of the body by securing a stent in a fixed configuration. However, the previous approaches due at least in part to retaining the stents in a fixed configuration and/or a reliance on elongation of the stent material itself may be prone to failure (e.g., stent migration), particularly over time due to repetition of natural body movements (e.g., organs shifting and moving due to peristalsis, respiration, or otherwise).

For instance, in various procedures where a stent extends across anatomical structures, such as to create an anastomosis, it may be desirable for the stent to remain in place for a prolonged period of time (e.g., days, weeks, months, even upwards of six to twelve months). However, due to natural body movements that stents positioned across anatomical structures (e.g., as compared to those merely deployed within a natural vessel) may be particularly prone to experience a greater quantity and/or larger magnitude of body movement (e.g., relative movement between two different organs which the stent is disposed between). Therefore the stents positioned across anatomical structures (e.g., even those which are formed of a flexible material) may be prone to stent migration.

As such, the approaches herein are directed to longitudinally self-expanding implantable medical devices that include a retention member located at the first end, the second end, or both the first end and the second end of the elongated body thereof. Notably, the retention member extends substantially traverse to the longitudinal axis of the implantable medical device and is configured to undergo deformation between a first configuration in an absence of an application of a force along a longitudinal axis of the implantable medical device and a second configuration responsive of the application of the force. Such deformation of the retention member can in turn translate to an change (e.g., an increase) in a longitudinal length of the longitudinally self-expanding implantable medical devices. For instance, the self-expanding devices can expand subsequent to implantation at a target regions in a vessel. That is, the approaches herein can mechanically deform such that the longitudinally expandable medical devices has a first length in a first (unexpanded) configuration and a second length in a second (expanded) configuration, where the second length is greater than the first length. This enhanced degree of longitudinal expansion is that is attributable at least to the deformation of the one or more retention structures herein is larger than a degree of longitudinal expansion permissible by employing a flexible material alone. Accordingly, the approaches herein can provide a greater degree of longitudinal expansion than previous approaches and thereby can accommodate natural body movement to mitigate stent migration even when the longitudinally expandable medical devices herein are deployed between different anatomical structures (e.g., as part of a CDS or HGS procedure).

Various embodiments of longitudinally expanding medical devices, systems, and methods in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

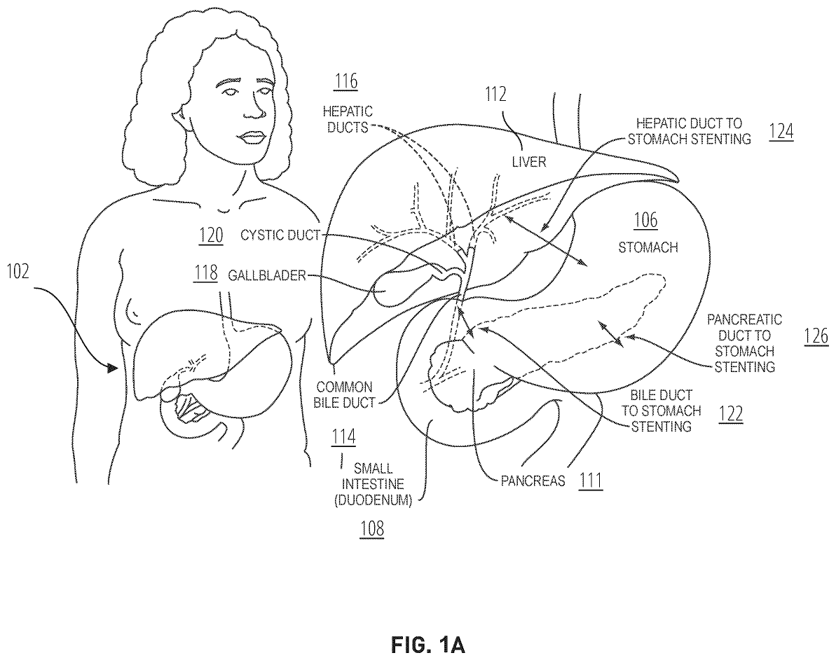

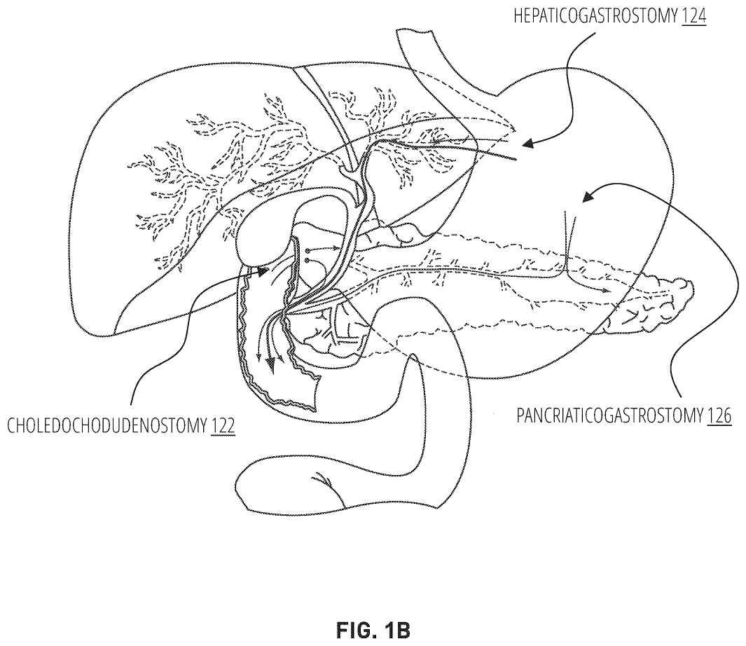

FIG. 1A and FIG. 1B illustrate examples of body lumens that can be connected by the stents disclosed herein. Areas within the abdominal cavity where stents described in this disclosure can be used to “span” or “connect” the common bile duct to the duodenum or the stomach to various positions in the biliary tree. Said differently, FIG. 1A and FIG. 1B illustrate various locations where stents can be placed within the abdominal cavity. In some embodiments, any of the stents disclosed herein can be placed in any of the locations illustrated in these figures. For example, any of the procedures illustrated in FIG. 1A or FIG. 1B can be used instead of an ERCP procedure. In some cases, an ERCP procedure can be unsuccessful or not possible, in those cases a stent can be placed through any of the pathways illustrated in FIG. 1A and FIG. 1B.

Turning more particularly to FIG. 1A, various locations within an abdominal cavity 102 of a patient 104 are depicted. For example, the stomach 106, duodenum 108, pancreas 111, liver 112, common bile duct 114, hepatic ducts 116, gallbladder 118, and cystic duct 120 are shown. Further, various stenting pathways are depicted.

For example, FIG. 1A and FIG. 1B depict a choledochodudenostomy 122, which connects the common bile duct 114 to the duodenum 108. For a choledochodudenostomy an endoscope can be advanced through the mouth and stomach 106 and into the duodenum 108. A target location in the common bile duct 114 can be identified using ultrasound guidance or other methods of guidance. A needle or catheter device can be advanced from the endoscope to puncture the wall of the duodenum 108 and the common bile duct 114. If a needle is used to access the common bile duct 114 then a guidewire can be placed with a catheter accessing the common bile duct 114 by advancing over the guidewire. The catheter can deploy a stent with an upstream end or flange within the common bile duct 114 and a downstream end or flange deployed in the duodenum 108 thereby forming a fluid conduit between the common bile duct 114 and the duodenum 108. In some instances, one or more interventions (e.g., vasculature dilation prior to stent deployment) may be performed prior to and/or subsequent to stent delivery and/or stent deployment.

As another example, FIG. 1A and FIG. 1B depict a hepaticogastrostomy 124, which connects the hepatic ducts 116 to the stomach 106. To perform a hepaticogastrostomy 124, an endoscope can be advanced through the mouth and into the stomach 106. The target location in the liver 112 can be identified using ultrasound guidance or other methods of guidance. A needle or catheter device can be advanced to puncture the stomach 106 and liver 112. A guidewire can be placed in the liver 112 (after needle access) followed by advancing a catheter carrying a stent over the guidewire to a target location (e.g., the hepatic ducts). An upstream end of the stent can be placed in the liver 112 and hepatic ducts 116 using the catheter. A downstream end of the stent is deployed within the stomach 106. The stent can have an uncovered portion on the end of the stent that is released inside the liver 112 and hepatic ducts 116. For example, the upstream end that is deployed within the liver 112 can have an uncovered portion of about 3-4 centimeters. The uncovered portion on the end of the stent can facilitate the flow of bile out of the liver and through the internal volume of the stent to drain to the stomach 106. The pressure in the liver 112 can assist the drainage of bile from the liver 112 through the stent and into the stomach 106. The downstream end of the stent deployed in the stomach 106 can be covered to reduce contact between the bile and the wall of the stomach 106. As mentioned, in some instances one or more interventions (e.g., vasculature dilation prior to stent deployment) may be performed prior to and/or subsequent to stent deployment. For instance, vasculature dilation may be performed (e.g., at a target location) subsequent to needle access and prior to stent delivery and/or stent deployment.

In another example, FIG. 1A and FIG. 1B depict a pancreaticogastrostomy 126, in which an endoscope can be advanced through the mouth and into the stomach 106. A target location (e.g., duct) in the pancreas 111 can be identified using ultrasound guidance or other methods of guidance. A needle or catheter device can be advanced from the endoscope to puncture the wall of the stomach 106 and the duct in the pancreas 111. A guidewire can be placed in the duct of the pancreas 111 (after needle access) followed by advancing a catheter carrying a stent over the guidewire. An upstream end of the stent can be placed in the duct of the pancreas 111 using the catheter. A downstream end of the stent is deployed within the stomach 106 thereby forming a fluid conduit between the duct in the pancreas 111 and the stomach 106.

In some embodiments, the stents disclosed herein can be used to place a stent anterograde. Anterograde stent placement can be done in the common bile duct 114 and ducts of the pancreas 111. Anterograde stent placement is where the operator enters the upstream part of the common bile duct 114 (or a duct in the pancreas 111). The upstream part of the common bile duct 114 can be accessed percutaneously (e.g., transhepatic) or under EDS-guidance (e.g., transenteric targeting an intra- or extra-hepatic bile duct). After obtaining access to the upstream part of the bile duct, a guide wire is inserted and advanced downstream to cross the stricture and ampulla and advanced into the duodenum 108. A stent is then advanced anterogradely over the wire to cross the stricture and the ampulla until the downstream end of the stent is in the duodenum 108. The sheath is retracted relative to the stent to release the downstream flange or double-walled flange. The sheath and stent can then be retracted as a single unit until the flange abuts against the ampulla of Vater, signaled by the resistance encountered with retraction. The sheath is then retracted relative to the stent to deploy the upstream flange inside the common bile duct 114. A similar procedure can be used to place a stent anterograde in ducts in the pancreas 111 after obtaining upstream access to the pancreas 111.

Moreover, although embodiments of the present disclosure are described with specific reference to medical devices and systems and procedures for treating biliary obstructions in the biliary system, it should be appreciated that such medical devices and methods may be used with implantable medical devices used in the abdominal cavity, digestive system, urinary tract, reproductive tract, respiratory system, cardiovascular system, circulatory system, etc.

FIG. 2A illustrates a view of an implantable medical device 220 in a first configuration (e.g., an undeformed configuration), while FIG. 2B illustrates a view of an implantable medical device 220 in a second configuration (e.g., deformed configuration) in accordance with various principles of the present disclosure. That is, the implantable medical devices herein such as the implantable medical device 220 can include retention members that are configured to elastically deform between first configuration in the absence of the application of a force along a longitudinal axis of the implantable medical devices and a second configuration responsive to the application of the force. As such, the implantable medical devices herein can permit enhanced variation in a length of the implantable medical devices (e.g., variation in a length of the saddle region) to account for forces imparted on the implantable medical devices while implanted and thereby mitigate any migration of the implantable medical devices.

As illustrated in FIG. 2A, the implantable medical device 220 includes an elongated body 224 having a first end 228, a second end 230, and a lumen 234 extending between the first end 228 and the second end 230. A saddle region 238 can be defined between the first end 228 and the second end 230. The length of the saddle region 238 can vary, as described herein, based on the deformation of a retention member 240.

The implantable medical devices herein can include a retention member located at the first end, the second end, or both the first end and the second end of an elongated body. For instance, as illustrated in FIG. 2A the implantable medical device 220 can include a retention member located at the second end 230 of the implantable medical device 220 and can have an absence of a retention member at the first end 228 of the implantable medical device 220. Having an implantable medical device 220 with a retention member such as the retention member 240 located at an individual end (e.g., the second end) of the medical device 220 can promote aspects herein such as providing a medical device that permits enhanced longitudinal movement of the implantable medical device 220 due at least in part to the presence of the individual member and yet, may yield a relatively small implantable medical device which can ease implantation (e.g., as compared to other medical device which include retention members at both the first and second ends thereof.

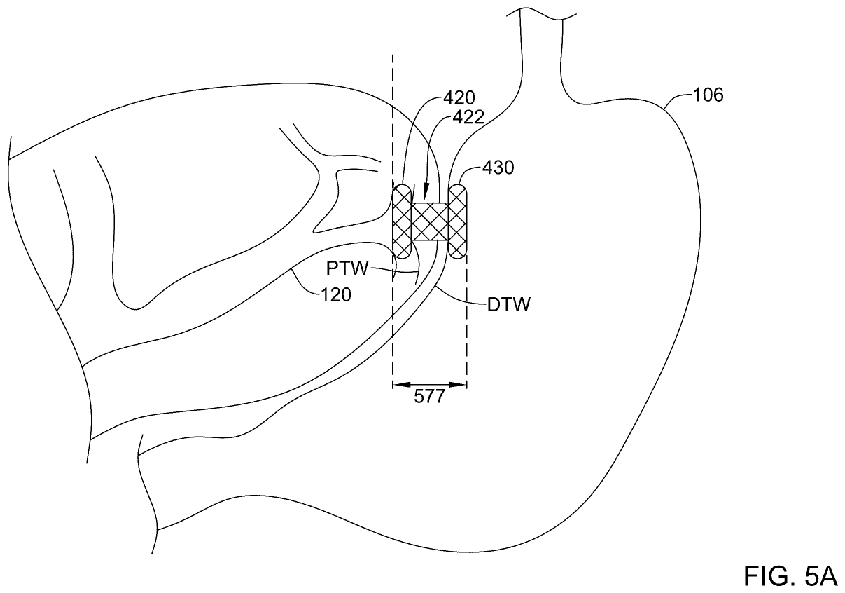

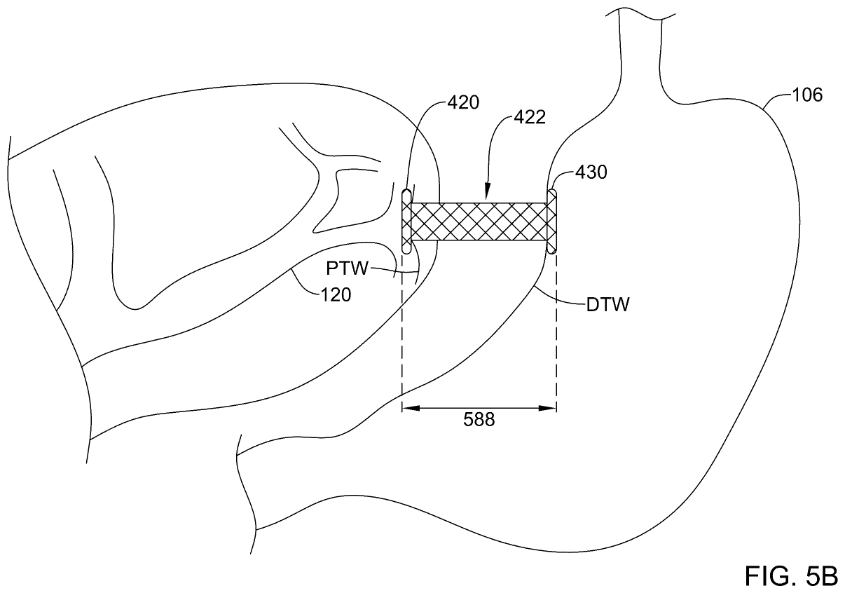

However, in some embodiments, the implantable medical devices can have a first retention member at the first end and a second retention member at the second end of the implantable medical devices. For instance, as described herein with respect to FIG. 5A the implantable medical device can include a first retention member at a second end thereof and a second retention member at a first end thereof. Having a respective retention member as each of the second end and the first end of the implantable medical devices can promote aspects herein such as providing an enhanced degree of deformation and thereby permitting an enhanced degree of longitudinal movement as compared to other approaches such as those are without a deformable retention member or employ an individual deformable retention member.

A retention member 240 can extend substantially traverse to the longitudinal axis of the implantable medical device 220, as illustrated in FIG. 2A. In some embodiments, the retention member 240 can be integral with the elongated body 224. That is, the retention member 240 can be integral with the saddle region 238. Having the retention member 240 be integral with the elongated body 224 (e.g., with the saddle region 238) can promote aspects herein. For instance, the retention members can be configured to undergo deformation and thereby vary a length (along the longitudinal axis of the implantable medical device 220) of the elongated body (e.g., the saddle region 238).

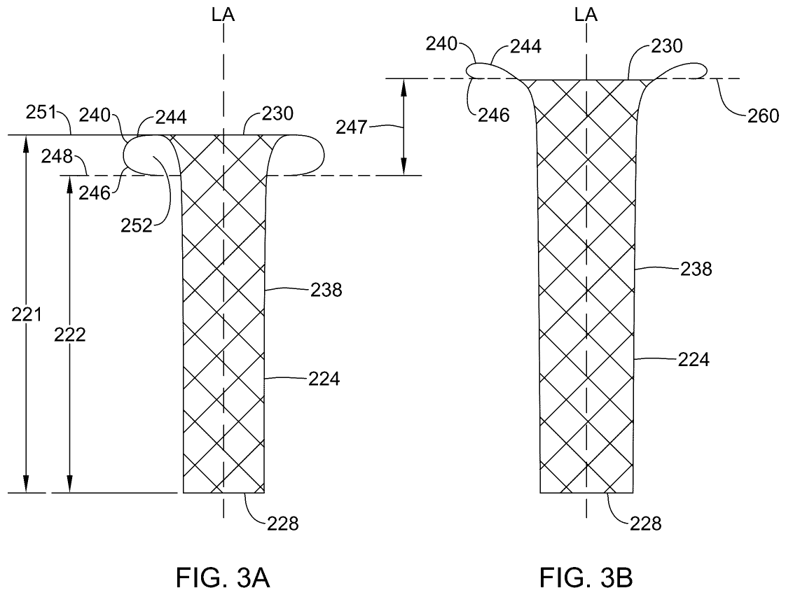

In some embodiments, the retention member 240 can have a substantially uniform surface (e.g., a substantially uniform surface formed of interwoven filaments) that is without a projection (e.g., a longitudinal projection) extending therefrom, as illustrated in FIGS. 2A-2B and 3A-3B. For instance, the retention member 240 may be manifested as a flange having a substantially uniform surface terminating in an arch or a rolled flange (e.g., terminating in one or more rolls of material). Having a substantially uniform surface on the retention members can promote aspects herein such as permitting the retention members to readily deform (e.g., roll or unroll) responsive to application or removal of a force on the implantable medical devices.

In some embodiments the retention members herein can have a longitudinal projection extending therefrom. For instance, as illustrated in FIGS. 2E, 2F, and 2G a plurality of projections 255 can extend (e.g., when in the second configuration) in a substantially longitudinal direction from the retention member 240. In some embodiments, the projections 255 can be formed of the same material as the retention member 240. For example, each of the projections 255 can be formed of one or more filaments such as one or more filaments that are configured in a loop. However, in some examples the projections 255 can be formed of a different material than the retention member 240. For instance, the projections 225 may be formed of a metal or other rigid material that is interwoven into filaments of the retention member 240.

Employing retention members that include the projections 255 can promote aspects herein such as providing an enhanced degree of resistance to a force (e.g., a longitudinal force) imparted on the retention member 240, for instance, when the retention member 240 is in the second configuration. For example, the longitudinal projections 255 can contact a tissue wall such as the tissue wall 223 when an implantable medical device 280 is in the second configuration, as illustrated in FIG. 2F, but can remain spaced a distance away from the tissue wall 223 when the retention member 240 is in the first configuration, as illustrated in FIG. 2E. For instance, the longitudinal projection 255 can remain in the gap 252 and be spaced a distance away from the tissue wall 223 when the implantable medical device 280 is in the first configuration. For instance, the longitudinal projection 255 can be positioned substantially at a midline or otherwise be located along a medial portion of the retention member 240 and can be disposed in the gap 252 when the retention member 240 is in a first configuration, as illustrated in FIG. 2E. In some instances, the plurality of projections 255 can be uniformly spaced (e.g., relative to each other and relative to the longitudinal axis of the implantable medical device 220), as illustrated in FIG. 2G.

In some embodiments, each of the components of the implantable medical device 220 can be formed of the same material. For instance, the retention member 240, the saddle region, and the elongated body 224 can be formed of the same material. In some embodiments, the retention member 240, the saddle region, and the elongated body can be formed of an interwoven stent filaments. As used herein, interwoven includes braided stent filaments, knitted stent filaments, and knotted stent filaments. In some embodiments, at least a portion of the implantable medical device 220 may be laser cut. Suitable materials for the stent filaments include alloys such as Elgiloy™ and Nitinol, and polymers such as polyethylene terephthalate (PET). The stent filaments may be cored or composite fibers, e.g., having a Nitinol outer shell and a platinum core. Some examples of cored or composite fibers are disclosed in U.S. Pat. No. 7,101,392, titled Tubular Medical Endoprostheses, and issued to Heath on Sep. 5, 2006; and U.S. Pat. No. 6,527,802, titled Clad Composite Stent, and issued to Mayer on Mar. 4, 2003, each of which patent is incorporated herein by reference in its entirety for all purposes.

As illustrated in FIG. 2A, the retention member 240 can include a flared region 244. The flared region 244 can be present when the implantable medical device 220 is in a first configuration. In some embodiments, the flared region 244 can terminate in an arch. For instance, the flared region 244 can terminate in arc 246 in the form of a concave arch (with respect to plane 248), as illustrated in FIG. 2A. Alternatively, the flared region 244 can terminate in a convex arch or other shape. For instance, the flared region 244 can terminate in an arch 246 in the form of a convex arch (with respect to plane 248), in a first (undeformed) configuration, as illustrated in FIG. 3A. That is, the flared region 244 can terminate in an arch or other substantially non-linear shape when the retention member is in a first configuration (e.g., as illustrated in FIG. 2A and FIG. 3A). Yet, the flared region 244 can deform responsive to the application of the longitudinal force to a substantially planar configuration (e.g., as illustrated in FIG. 2B and FIG. 3B), thereby increasing a length (along the longitudinal axis) of the implantable medical devices herein. For instance, in the second configuration the plane 248 extending along the tissue wall 223 and the plane extending 251 along the second end 230 of the implantable medical device 220 can form a substantially common plane 260 including both the plane 248 and the plane 251, as illustrated in FIG. 3B.

While FIG. 2A and FIG. 3A illustrate the flared region 244 as terminating in an arch, other configurations are possible. For instance, the flared region 244 can terminate in a rolled flange, as illustrated in FIG. 4A. In any case, having the flared region terminate in an arch or a rolled flange can promote aspects herein such as permitting enhanced longitudinal movement of the implantable medical devices herein.

A portion of the flared region 244 can contact a tissue wall 223, as illustrated in FIG. 2A. For instance, the flared region 244 can therefore be configured to anchor the implantable medical device 220 in contact with the tissue wall 223. When in the first configuration, the flared region 244 of the retention member 240 can maintain a gap 252 (e.g., a longitudinal gap) between a plane 248 extending along the tissue wall 223 and a plane extending 251 along the second end 230 of the implantable medical device 220. The gap 252 can be equal to or can be substantially equal to a difference between a second length 222 and the first length 221 of the implantable medical device 220. Having the gap 252 present when in the first (unexpanded) configuration can thereby facilitate a greater degree of longitudinal movement of the implantable medical device 220.

For instance, responsive to application of a longitudinal force to the implantable medical device 220 the flared region 244 of the retention member 240 can translate toward the tissue wall 223 and thereby reduce or eliminate the gap 252 between the plane 248 extending along the tissue wall 223 and the plane 251 extending along the second end 230 of the implantable medical device 220. The reduction or elimination of the gap 252 responsive to the application of the longitudinal force can increase a length (along the longitudinal axis) of the medical device 220. The increased longitudinal length 247 of the medical device 220 can entirely or substantially attributable to the reduction or elimination of the gap 252 responsive to the application of the longitudinal force. For instance, in the second configuration the plane 248 extending along the tissue wall 223 and the plane extending 250 along the second end 230 of the implantable medical device 220 can extend along a common plane 260, as illustrated in FIG. 2B.

The retention member 240 can be configured to undergo deformation between a first configuration (e.g., as illustrated in FIG. 2A with an absence of an application of a force along a longitudinal axis of the implantable medical device 220) and a second configuration (e.g., as illustrated in FIG. 2B) responsive of the application of the force along the longitudinal axis of the medical device. As mentioned, the force may be imparted due to that natural movement or shifting of organs within a body. For instance, the retention member 240 can have a first (unexpended) length 221 when in the first configuration in FIG. 2A and can have a second (longitudinally extended) length 222 when in the second configuration.

As illustrated in FIG. 2A the first length 221 extends along the longitudinal axis (LA) of the longitudinally expandable implantable medical device 220. Similarly, the second length extends along the longitudinal axis (LA). The second length 222 can be longer than the first length 221. Such variation in the length of the elongated body 224 (e.g., the saddle region 238) can permit the implantable medical device 220 to accommodate (e.g., expand or contract alone a longitudinal axis) for bodily movement between different organs, etc. That is, the implantable medical devices herein at least due to the presence of the retention members can undergo a degree of longitudinal expansion that is greater than other approaches such as those which include sutures, locking mechanisms, and/or are otherwise configured to retain an implanted device in a fixed location (e.g., with a fixed longitudinal length) and/or those that merely employ a flexible (e.g., shape memory) material. For example, some previous approaches may employ a flexible (e.g., shape memory material) in an implantable device that is otherwise fixed with a given longitudinal length. Such approaches may be not exhibit a degree of longitudinal extension (e.g., about one millimeter or less, etc.) sufficient to accommodate a degree of natural body movement between different organs and thus such devices may be prone to migration and/or failure (e.g., elastic failure of the flexible material).

Accordingly, the self-expanding implantable devices herein permit a greater degree of longitudinal movement between a first length (e.g., in a first configuration) and a second length (e.g., in a second configuration). For example, a difference between the second length and the first length can be in a range from about 5 millimeters to about 2 centimeters. All individual values and sub-ranges from 5 millimeters to about 1 centimeter are included. For instances, the difference between the second length and the first length can be greater than about 5 millimeters, greater than about 6, greater than about 7 millimeters, greater than about 8 millimeters, greater than about 9 millimeters, greater than about 10 millimeters, greater than about 11 millimeters, greater than about 12 millimeters, greater than about 13 millimeters, greater than about 14 millimeters, greater than about 15 millimeters, greater than about 16 millimeters, greater than about 17 millimeters, greater than about 18 millimeters, greater than about 19 millimeters, or greater than about 20 millimeters, among other possible values.

FIG. 2C illustrates a section view (taken along plane 260 in FIG. 2B) of the implantable medical device 220. As illustrated in FIG. 2C, the flared region 244 can extend circumferentially about the implantable medical device 220. For instance, the flared region 244 can be formed of filaments such as those described herein that extend radially from and circumferentially about an elongated body of the implantable medical device 220, as illustrated in FIG. 2C. In some embodiments, the flared region 244 can be formed of woven or braided filaments that are the same shape, size, and filament density (e.g., have the same quantity of filaments and/or same quantity of filament intersections per area), as illustrated in FIG. 2C.

However, in some embodiments, the flared region 244 can be formed of filaments that are different shapes, sizes, and/or filament densities to provide the flared region 244 with a plurality of sub-regions having different properties. For instance, as illustrated in FIG. 2D a first (radial-most) region 270 can be configured to have a relatively high stiffness (e.g., axial stiffness) and a second region 272 which is more proximate to the longitudinal axis of the implantable medical device 220 can be configured with a relatively lower stiffness (e.g., lower axial stiffness), among other possibilities. For instance, the first region 270 can include a larger quantity of filaments and/or larger quantity of filament intersections per area. While the first and second regions are illustrated as being distinct regions in FIG. 2D, in some instances the flared region 244 can include a material included in an radial gradient of increasing or decreasing axial stiffness.

FIG. 4A illustrates a view of an implantable medical device 422 in a first configuration in accordance with various principles of the present disclosure, while FIG. 4B illustrates a view of an implantable medical device 422 in a second configuration in accordance with various principles of the present disclosure. The example of an embodiment of an implantable medical device 422 illustrated in FIG. 4A includes an elongated body 410 having a first end 411 and a second end 413. The elongated body 410 of the implantable medical device 422 may generally have a tubular configuration with a lumen 415 extending therethrough, such as between the first end 411 and the second end 413 thereof. The elongated body 410 may extend the full length of the implantable medical device 422 (e.g., the first end 411 and the second end 413 of the elongated body 110 may be substantially coextensive with the first end 411 and the second end 413, respectively, of the implantable medical device 422), or may extend only a portion of the full extent or length of the implantable medical device 422, the present disclosure not being limited in this regard.

The implantable medical device 422 typically is shiftable between a delivery configuration and a deployed configuration. In the delivery configuration, size, shape, configuration, and/or dimensions of the implantable medical device 422 facilitate transluminal delivery thereof (e.g., through natural body passages) to an anatomical site. In the deployed configuration, the implantable medical device 422 may be sized, shaped, configured, and/or dimensioned to achieve various structures or forms for various purposes, such as to facilitate positioning of the implantable medical device 422 with respect to a treatment site, anchoring with respect thereto, formation of a passage through the anatomical site, supporting of tissue and/or tissue walls, etc. For instance, when in the delivery configuration, the elongated body 410 may have an elongated length (along a longitudinal axis LA thereof) and/or a reduced-diameter (generally transverse to the longitudinal axis LA) relative to the deployed configuration. In some embodiments, the elongated body 410 may be considered to be in a constrained, unexpanded, constricted, restrained, collapsed, etc., configuration when in the delivery configuration (not shown, but which may readily be appreciated by those of ordinary skill in the art). In the deployed configuration, the elongated body 410 may have a generally foreshortened and/or radially expanded configuration, such as relative to the delivery configuration. In some embodiments, the elongated body 410 may be considered to be in an unconstrained, expanded, unconstricted, unrestrained, neutral, etc., configuration when in the deployed configuration.

In accordance with various principles of the present disclosure, the implantable medical device 422 includes a first retention member 420 along a first end 411 of the implantable medical device 422 and a second retention member 430 along a second end 413 of the implantable medical device 422. It will be appreciated that although one retention member is formed along each end of the implantable medical device 422, more than one retention member may be provided on either or both ends of the implantable medical device 422. In some embodiments, a portion of the first end 411 of the elongated body 410 radially expands to form the first retention member 420, and a portion of the second end 413 of the elongated body 410 radially expands to form the second retention member 430, defining a saddle region 440 extending therebetween. In the deployed configuration of the implantable medical device 422, the saddle region 440 typically has a diameter greater than the diameter of the elongated body 410 in the delivery configuration. The retention members 420, 430 generally have respective diameters larger than the diameter of the saddle region 440. The diameters of the retention members 420, 430 may be the same as or different from each other, such as depending on the intended use of the implantable medical device 422 as may be appreciated by those of ordinary skill in the art.

The retention members 420, 430 may be sized, shaped, configured, and/or dimensioned to anchor the implantable medical device 422 with respect to tissue walls (e.g., extending radially outwardly from a body passage through which the saddle region 440 of the implantable medical device 422 extends) to resist migration of the implantable medical device 422 with respect to the deployment site. For instance, in some embodiments the retention members 420, 430 can each be the same shape, the same size, and/or be formed of the same material. For example, the retention members 420, 430 can be rolled retention members that are the same shape, the same size, and formed of the same material, but are configured in opposing directions with respect to the longitudinal axis of the implantable medical device 422, as detailed herein.

In some embodiments, at least a portion of one or both of the retention members 420, 430 is angled towards the saddle region 440 or otherwise includes a portion or surface which protrudes towards the saddle region 440 to exert pressure against the tissue wall against which the retention members 420, 430 are positioned. For instance, the retention members 420, 430 can be rolled retention members, as illustrated in FIGS. 4A-4B. The rolled retention members can have a substantially circular cross-sections and be formed of one or more rolls of material that form the retention members 420, 430. For instance, the rolled retention members can be formed of rolled material that is rolled (e.g., coiled) along an axis that is coaxial with the longitudinal axis of the implantable medical device 422. In some instances, the rolled retention members can be rolled in opposing directions with respect to each other and/or with respect to the longitudinal axis of the implantable medical device 422. For instance, each of the rolled retention members can be rolled about a respective axis such that each of the rolled retention members includes material rolled toward the saddle region 440. Having each of the rolled retention members include material rolled in opposing directions can promote aspects herein such as permitting the enhanced longitudinal expansion of the implantable medical device 422 along a longitudinal axis of the implantable medical device 422 responsive to a longitudinal force, as detailed herein.

In some embodiments, the rolled retention members (e.g., the rolled retention members 420, 430) can include a plurality of anti-migration features 480, 482 extending therefrom. The anti-migration features 480, 482 may be sized, shaped, configured, dimensioned, positioned, located, etc., in a variety of manners to increase resistance of the implantable medical device 422 against migration from the implant site, which may be appreciated by those of ordinary skill in the art in view of the following. Typically, the anti-migration features 480, 482 are provided along an outer surface of the implantable medical device 422 which faces tissue so that the anti-migration features 480, 482 interact with the tissue to hold the implantable medical device 422 with respect to the tissue to resist migration therefrom.

The anti-migration features 480, 482, may extend transverse to a direction of potential migration of the implantable medical device 422 and/or transverse to the primary direction in which forces may impact the implantable medical device 422. In the examples of embodiments illustrated in FIG. 4A, and FIG. 4B, the anti-migration features 480, 482 extend transverse to a direction of potential migration of the implantable medical device 422 and/or transverse to the primary direction in which forces may impact the implantable medical device 422. In the examples of anatomical sites illustrated in FIG. 1A-1B, the implantable medical device 422 is typically subjected to the greatest forces (within the anatomical site at which it is deployed) along the longitudinal axis LA thereof. Accordingly, as may be appreciated with reference to the example of an embodiment of an implantable medical device 422 illustrated in FIGS. 4A-4B, the anti-migration features 480, 482 extend generally transverse to the longitudinal axis LA of the implantable medical device 422. Other configurations (e.g., in-line with or coaxial with the longitudinal axis of the implantable medical device 422) are possible. The anti-migration features 480, 482 herein (e.g., teeth formed of one or more filaments terminative in a triangular or other shape having a pointed end) can immediately contact and engage with a tissue wall and thereby mitigate migration of the implantable medical device without having to rely only on the relatively slower process of tissue in-growth that some other approaches (e.g., those employing U-shaped or looped anti-migration features). Yet, the approaches herein can also promote tissue in-growth such as tissue in-growth along an interface between the anti-migration feature 480, 482 and a tissue wall and thereby can yield a further mitigation in any migration of the implantable medical device from an anatomical site at which the implantable medical device is implanted.

In some aspects, the anti-migration features 480, 482 are not coated or are otherwise not provided with a coating as provided on other portions of the implantable medical device 422, particularly along the saddle region 440). In accordance with various principles of the present disclosure, the uncoated anti-migration features 480, 482 are provided along one or more portions or areas of the implantable medical device 422 facing tissue at the deployment site. The lack of coating on the anti-migration features 480, 482 allows and may even promote tissue ingrowth around the anti-migration features 480, 482 so that the implantable medical device 422 is held in place with respect to the deployment site.

For instance, a first plurality of anti-migration features 480 can extend substantially longitudinally from an end of a first rolled retention member (e.g., the retention member 420) and being configured to anchor the implantable medical device 422 with respect to the first tissue wall (e.g., a proximal or distal tissue wall) and a second plurality of anti-migration features 482 extending substantially longitudinally from an end of the second rolled retention member and being configured to anchor the implantable medical device with respect to the second tissue wall (e.g., the other of the proximal or distal tissue wall). As illustrated in FIGS. 4A-4B the anti-migration features can extend in opposing directions. For instance, the first plurality of anti-migration features 480 can extend in first direction along the longitudinal axis and/or coaxial with the longitudinal axis of the implantable medical device 422 and the second plurality of anti-migration features 482 can extend in a second direction along the longitudinal axis and/or coaxial with the longitudinal axis of the implantable medical device 422 that is opposite the first direction, as illustrated in FIG. 4A-4B. Accordingly, the first rolled retention member 420 can be configured to unroll in a first direction about or coaxial with the longitudinal axis of the implantable medical device 422 responsive to the application of a force (e.g., a longitudinal force) and the second rolled retention member 430 can configured to unroll in a second direction about or coaxial with the longitudinal axis that is opposite from the first direction. Having the anti-migration features 480, 482 extend in opposing directions can promote aspects herein such as promoting anchoring of the respective anti-migration features against a respective tissue wall and thus can further mitigate any migration of the implantable medical device 422.

In some embodiments, the first plurality of anti-migration features 480 comprise a plurality of teeth that are disposed uniformly about a substantially circular end of the second rolled retention member 413 and the second plurality of anti-migration features 482 comprise teeth that are disposed uniformly about a substantially circular end of the first rolled retention member 411. In some embodiments, the first plurality of anti-migration features 480 and the second plurality of anti-migration features 482 comprise teeth that are the same shape and size, but are orientated in opposing directions, as illustrated in FIG. 4A-4B. The teeth may be formed of filaments of the terminating ends or one or more filaments that form the elongated body 410 of the implantable medical device 422. That is, the rolled flanges may terminate in the anti-migration features (e.g., teeth), as illustrated in FIGS. 4A-4B.

The retention members 420, 430 may be formed as single-wall structures or double-wall structures. For instance, if the retention members 420, 430 are formed by expansion of first (e.g., proximal) and/or second (e.g., distal portions) of the wall of the implantable medical device 422/elongated body 410, such expanded wall(s) may extend radially outwardly and then return radially inwardly to form a double-wall retention member 420, 430. It will be appreciated that the retention members 420, 430 need not be limited to being a part of the ends of the elongated body 410 and may additionally or alternatively be considered extensions of the elongated body 410 at ends of the implantable medical device 422, the present disclosure not being limited in this regard.

In some embodiments (such as the example of an embodiment illustrated in FIG. 4A), at least a portion of the implantable medical device 220 may advantageously be coated with a material which maintains flow of materials (such as fluids) through the lumen 115 defined through the elongated body 410 without flowing transversely through at least portions of the walls of the elongated body 410. For instance, in embodiments in which the implantable medical device 220 forms a flow passage, it may be desirable to restrict flow of materials to through the lumen 415 from the first end 411 to the second end 413 of the implantable medical device 422 without leakage through/out of the lumen 415 (e.g., through the walls of the implantable medical device 422). In some embodiments, the coating is at least over portions of or the entire saddle region 440 of the implantable medical device 422 to maintain flow of materials through the saddle region 440 without flowing through the wall thereof. In some embodiments, the coating is also over additional portions of the implantable medical devices, such as over at least portions of or all of one or both of the retention members 420, 430. It will be appreciated that the retention members 420, 430 may include respective lumens defined therethrough as well (such as extensions of the lumen 415 defined through the elongated body 410), and the retention members 420, 430 may also be coated with a material which maintains flow of materials (such as fluids) through the lumens thereof (and not through the walls of the retention members 420, 430) to flow through the lumen 415 through the elongated body 410. The coating may also provide a degree of structural stability or rigidity to the implantable medical device 422. The coating may also mitigate ingrowth in the retention members 420, 430 and thereby promote ease of the retention members 420, 430, deforming between a first (unexpanded) configuration and a second (expanded) configuration, as described herein.

The coating may be provided on the implantable medical device 422 (the entire implantable medical device 422 or portions thereof) in any of a variety of manners, such as painting, dipping, spraying, sandwiching, heat shrinking, electrospinning, etc. The coating may be provided on only the outer surface or only the inner surface or both the outer surface and the inner surface of at least portions of the implantable medical device 422 wall. The coating may be any known or heretofore known biocompatible material which may prevent flow of fluids therethrough, including, without limitation, silicone, styrene isoprene butadiene (SIBS), polytetrafluoroethylene (PTFE), expanded polytetrafluoroethylene (ePTFE), ethylene tetrafluoroethylene (ETFE), fluorinated ethylene propylene (FEP), urethane, polyurethane, polyvinylidene chloride (PVC), polyether block amides (PEBA), polyimide, polyethylene, polyethylene terephthalate (PET), polysulfone, nylon, polytrimethylene terephthalate, polyvinylidene difluoride (PVDF), polyester, polyether-ester, polypropylene, polyolefin, polystyrene, polynapthalene, polyethylene napthalate (PEN), polyetherether ketone (PEEK), polyetherimide, polyphenylene sulfide (PPS), polyphenylene oxide (PPO), perfluoro(propyl vinyl ether) (PFA), polyparaphenylene teraphthalamide, polybutylene terephthalate (PBT), polyoxymethylene (POM), polyether block ester, poly(styrene-butadiene-styrene) (SBS), styrene-ethylene-butylene-styrene (SEBS), poly(styrene-b-isobutylene-b-styrene), ethylene vinyl alcohol, ethylene vinyl acetate copolymers (EVA), polycarbonates, ionomers, thermoplastic elastomers (TPE), epoxy, etc., including copolymers and/or combinations thereof.

In some embodiments, the coating may be applied to an entire implantable medical device 422 and subsequently may be selectively removed from a portion of the implantable medical device 422. For instance, the coating may be applied to an entire implantable medical device 422 and may be subsequently selectively removed (e.g., with a laser and/or masking/etch, etc.) to expose uncoated anti-migration features 480, 482, (e.g., uncoated teeth) on the implantable medical device 422.

The implantable medical devices herein may be formed in a variety of manners, such as to form a scaffold or stent structure. In some embodiments, the implantable medical devices herein formed from one or more members/elements (such terms being used interchangeably herein without intent to limit) combined to form a rigid and/or semi-rigid structure. The implantable members herein may be formed of one or more struts, wires, strands, filaments, etc., which are braided, interengaged, intertwined, interwoven, knitted, knotted, looped (e.g., bobbinet-style), weaved, woven, wrapped, or the like to form an expandable and contractable scaffold configuration. For the sake of convenience, and without intent to limit, reference is made to filaments which are woven to form the wall of the implantable medical devices herein. The filaments forming the implantable medical device herein may be formed from a variety of non-limiting preferably biocompatible materials, such as, without limitation, a metal, metal alloy, polymer, metal-polymer composite, ceramics, and combinations or sub-combinations thereof. For instance, the filaments forming the implantable medical devices herein may be formed from a variety of non-limiting preferably biocompatible polymers, such as, without limitation, polypropylene, polyester, polysulfone, nylon, silicones, polyurethane, polystyrene, polyethylene (PE) (including high-density and low-density PE's), polyethylene terephthalate (PET), polyethylene naphthalate (PEN), polybutylene terephthalate (PBT), polytrimethylene terephthalate, polyether block amides (PEBA), polyetheretherketone (PEEK), polyetherimide (PEI), poly(methyl methacrylate) (PMMA), polytetrafluoroethylene (PTFE), ethylene tetrafluoroethylene (ETFE), fluorinated ethylene propylene (FEP), polyoxymethylene (POM), polyether block ester, polyvinylchloride (PVC), polyvinylidene chloride (PVDC), polyether-ester, ether or ester based copolymers (for example, butylene/poly(alkylene ether) phthalate and/or other polyester elastomers, polyamides, block polyamide/ethers, polyimide (PI), ethylene vinyl alcohol, ethylene vinyl acetate copolymers (EVA), polyphenylene sulfide (PPS), polyphenylene oxide (PPO), poly paraphenylene terephthalamide, perfluoro(propyl vinyl ether) (PFA), polyolefin, epoxy, poly(styrene-b-isobutylene-b-styrene), polycarbonates, ionomers, or the like including mixtures, combinations, sub-combinations, and copolymers thereof. Additionally or alternatively, the members forming the implantable medical device may be formed from a variety of non-limiting preferably biocompatible metals, such as, without limitation, stainless steel, a nickel-titanium alloy such as Nitinol, a cobalt-chromium alloy, a cobalt-chromium-nickel based alloy such as Elgiloy®, a nickel-cobalt alloy, a nickel-iron alloy, a nickel-chromium alloy, a nickel-molybdenum alloy, a nickel-chromium-molybdenum alloy, a nickel-cobalt-chromium-molybdenum alloy, a cobalt-chromium-molybdenum alloy, platinum enriched stainless steel, titanium, or the like, including combinations and sub-combinations and other alloys thereof. Additionally or alternatively, the members forming the implantable medical devices herein may be formed from a variety of non-limiting preferably biocompatible natural materials, such as, without limitation, cat or bovine intestine, or the like; a natural fiber, such as silk or cotton, or the like, and combinations and sub-combinations thereof. It will be appreciated that the members forming the implantable medical devices herein may be formed from a mixtures, composites, combinations, sub-combinations, copolymers, or co-constructions of any of the above. Alternatively, the members forming the implantable medical devices herein may be formed by cutting (e.g., by laser-cutting) a tubular structure (e.g., an, optionally monolithic, cylindrical tubular member) into an expandable configuration, the cuts forming members such as strut members. The implantable medical devices herein may be a self-expanding device such as known or heretofore known to those of ordinary skill in the art. For instance, the implantable medical device may be formed of shape-memory or heat-formable material (e.g., Nitinol or Elgiloy® or shape memory polymers) so that the implantable medical devices herein return to a pre-shaped expanded configuration from a collapsed configuration upon advancement from a delivery sheath (any acceptable tubular elongated member such as known to those of ordinary skill in the art for delivery of medical devices) and/or withdrawal of a delivery sheath which maintains the implantable medical devices in a delivery configuration therein.

FIG. 5A depicts the implantable medical device 422 of FIG. 4A) deployed in contact with anatomical tissue in a hepaticogastrostomy in a first (unexpanded) configuration, while FIG. 5B depicts the implantable medical device in a second (expanded configuration). FIGS. 5A-5B illustrate the medical device 422 as including two retention members 420, 430 to illustrate various aspects of the disclosure. For instance, as illustrated in FIGS. 5A-5B, the implantable medical device 422 can have a first length 577 in the first configuration and a second length 588 in the second configuration, where the second length 588 is greater than the first length 577, as detailed herein. However, as detailed herein in some embodiments a medical device can include an individual retention member. Stated differently, in some embodiments a medical device can be provided that includes a retention member located only at a second end or a first end of the medical device, but does not have a retention member located at the other of the second end or the first end of the medical device. For instance, in some embodiments a medical device can include an individual retention member located at a first end and includes a second end that is retention member free (does not include a retention member), as described herein. However, in some embodiments, a medical device can include an individual retention member located at a second end and includes a first end that is retention member free (does not include a retention member, as described herein.

In some embodiments, such as illustrated in FIG. 5A, the one or more anti-migration features 480, 482 extend from a radially-extending wall 423, 433 of at least one of the retention members 420, 430. As referenced herein, the radially-extending walls 423, 433 are the walls forming the retention members 420, 430 which are generally transverse to the longitudinal axis LA of the implantable medical device 422. The radially-extending walls 423, 433 of the retention members 420, 430 are generally also transverse to the saddle region 440 which generally extends along the longitudinal axis LA of the implantable medical device 422. Such configuration is advantageous when the implantable medical device 422 is deployed at anatomical sites where forces within the body impact the implantable medical device 422 along the longitudinal axis LA thereof. For instance, if the implantable medical device 422 defines a flow passage therethrough (e.g., through a lumen (e.g., lumen 415 as illustrated in FIG. 4A), forces which may cause migration of the implantable medical device 422 are typically greatest along the longitudinal axis LA thereof. Thus, the radially-outwardly extending walls of the retention members 420, 430, as transverse to the longitudinal axis LA of the implantable medical device 422, provide increased surface area to resist axial forces on the implantable medical device 422. The radially-outwardly extending walls of the retention members 420, 430 may anchor implantable medical device 422 against the tissue walls extending (e.g., radially outwardly) from the passage through which the implantable medical device 422 is deployed and/or the tissue walls against which the retention members 420, 430 are deployed. In some embodiments, the anti-migration features 480, 482 are positioned along the radially outermost edge of the retention members 420, 430. Such position of the anti-migration features 480, 482 may be beneficial in contributing to retaining the greatest surface area of the radially-extending walls 423, 433 of the retention members 420, 430 being pressed against tissue. However, other locations are within the scope and spirit of the present disclosure.

The anti-migration features 480, 482 may extend substantially against or along or parallel to or generally flat with respect to the wall of the implantable medical device 422 when the implantable medical device 422 is in a delivery configuration to allow a compact configuration thereof. However, in a deployed configuration of the implantable medical device 422, the anti-migration features 480, 482 may extend at various angles (i.e., angles greater than 0 degrees and less than 180 degrees) with respect to the wall of the implantable medical device 220 from which the anti-migration features 480, 482 extend. For instance, the anti-migration features 480, 482 may extend from a wall of the implantable medical device 422 in a direction transverse to such wall. For instance, in the example of an embodiment illustrated in FIG. 4A, the anti-migration features 480, 482 extend from and generally transverse to the respective radially-extending walls 423, 433 of the retention members 420, 430 along the side of the retention members 420, 430 facing towards the saddle region 440. Thus, the anti-migration features 480, 482 extend from the retention members 420, 430 towards the saddle region 440.

In some embodiments, the anti-migration features 480, 482 may be configured to extend into the walls of the anatomical tissue along which the implantable medical device 422 is deployed. It will be appreciated that terms such as penetrate, anchor, bite, embed, etc., and other grammatical forms thereof, may be used interchangeably herein without intent to limit. As illustrated in FIGS. 5A-5B, an example of an embodiment of an implantable medical device 422 is extended between a first (e.g., proximal) tissue wall PTW and a second (e.g., distal) tissue wall DTW. Specifically, the elongated body 410 of the implantable medical device 422 extends from a first (e.g., proximal) side of the first (e.g., proximal) tissue wall PTW to the second (e.g., distal) side of the second (e.g., distal) tissue wall DTW with the first (e.g., proximal) retention member 420 positioned against the first (e.g., proximal) side of the first (e.g., proximal) tissue wall PTW and the second (e.g., distal) retention member 430 positioned against the second (e.g., distal) side of the second (e.g., distal) tissue wall DTW. In the illustrated example of an embodiment, anti-migration features 480, 482 extend transversely from the retention members 420, 430 and towards the saddle region 440 of the implantable medical device 422. The length of the elongated body 410, particularly of the saddle region 440 thereof, may be selected so that the retention members 420, 430 exert pressure against the tissue walls PTW and DTW to hold the tissue walls PTW and DTW in apposition. Such pressure may cause the anti-migration features 480, 482 to embed into the tissue walls PTW and DTW to further enhance the anti-migratory nature of the anti-migration features 480, 482. As the tissue walls PTW and DTW are held in apposition, tissue may grow along the saddle region 440 of the implantable medical device 422 to form an anatomical/tissue (as opposed to an artificial, such as formed by the implantable medical device 422) anastomosis. In some instances, tissue growth along the saddle region 440 may even be encouraged, particularly if pressure exerted by the retention members 420, 430 (enhanced by the anti-migration features 480, 482) is exerted against the apposed tissues, such as causing the apposed tissues to fuse together. In some embodiments, the plurality of anti-migration features 480, 482 are teeth that are configured to contact a tissue wall (e.g., the tissue walls PTW and DTW) when the retention member is in a first configuration and remain in contact with the tissue wall when the retention member is in a second configuration, as illustrated in FIGS. 5A-5B.

It will be appreciated that the anti-migration features 480, 482 may be formed in any of a variety of manners in accordance with various principles of the present disclosure. The anti-migration features 480, 482 may be formed as wires or filaments or threads or tethers or ropes or bands or other elements providing sufficient resistance to forces impacting the implantable medical device 422 and the anti-migration features 480, 482. The anti-migration features 480, 482 may be formed separately from the implantable medical device 422 and coupled (directly or indirectly) thereto, such as by welding, soldering, interweaving, adhering (e.g., gluing). mechanically deforming (e.g., knotting, looping, crimping, interference or friction fitting, etc.), or other manners known to those of ordinary skill in the art. Additionally or alternatively, the anti-migration features 480, 482 may be integral extensions of the walls of the implantable medical device 422. For instance, in some embodiments, one or more of the elements forming the implantable medical device 422 (e.g., woven or interwoven filaments) may be extended or pulled away from the remainder of the implantable medical device 220 to form an anti-migration feature.

In view of the above descriptions, it will be appreciated that the devices, systems, and methods disclosed herein can be used to form one or more anastomoses, and can be used with basic endoscopic tools, catheters, laparoscopes, general surgery tools, etc. For example, a catheter-based stent delivery device can be used with an endoscope to form one anastomosis. When deploying a stent or other tissue anchor between adjacent body lumens, organs, or other structures, it is typically necessary to penetrate both a first tissue wall (e.g., a wall of an organ or a first body lumen), through which access is established, and a second tissue wall (e.g., of a wall of an organ or a second body lumen) which is the target for the procedure. For instance, an instrument may be introduced to an anatomical site at which the anastomosis is to be performed to form (e.g., cut) a passage between adjacent tissues. Tissue at the deployment site may be pretreated in a variety of manners, such as by abrasion (e.g., with the use of a hook knife, hot biopsy forceps, and hot snares), ablation, pharmaceutical treatments, argon plasma coagulation (APC), etc., to promote, accelerate, and/or increase cellular growth, such as a result of a healing response. The induced tissue growth may facilitate the above-described tissue ingrowth into the anti-migration features of the implantable medical device to be positioned at the treated site. A delivery device (e.g., tubular elongate member) may then be navigated to the anatomical site with the implantable medical device therein. The second end may be extended from the delivery device and/or the delivery device may be retracted to deploy a second end of the device. In some embodiments, the second end expands to form a second retention member anchoring the implantable medical device with respect to the second (e.g., distal) tissue wall. The delivery device may then be further withdrawn to expose the portions of the implantable medical device proximal to the second end of the implantable medical device. The first end of the implantable medical device may expand to form a first (e.g., proximal) retention member anchoring the implantable medical device with respect to the first (e.g., proximal) tissue wall.

It will be appreciated that the present disclosure is not to be limited to a particular form or configuration of an implantable medical device, or system or method used therewith, principles of the present disclosure being applicable to various configurations of implantable medical devices, systems, and methods such as known to those of ordinary skill in the art. It will be appreciated that various aspects of the above disclosure may be applied to other implantable medical devices, systems, and/or methods, such as devices positioned in other locations within the body, whether or not a flow passage exists or is created at such location.

Various further benefits of the various aspects, features, components, and structures of anti-migration features such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An implantable medical device comprising:
an elongated body having a first end, a second end, and a lumen extending therebetween;
a saddle region defined between the first end and the second end;
a retention member at the first end, the second end, or both, wherein the retention member extends substantially traverse to a longitudinal axis of the implantable medical device and is configured to deform responsive to an application of a force along a longitudinal axis of the implantable medical device.

2. The implantable medical device of claim 1, wherein the retention member is configured to elastically deform between:
a first configuration in an absence of the application of the force; and
a second configuration responsive to the application of the force.

3. The implantable medical device of claim 1, wherein the retention member, the saddle region, and the elongated body are formed of the same material.

4. The implantable medical device of claim 3, wherein the material is a shape memory material.

5. The implantable medical device of claim 1, wherein the retention member is integral with the saddle region.

6. The implantable medical device of claim 1, wherein the retention member includes a flared region.

7. The implantable medical device of claim 6, wherein the flared region terminates in an arch.

8. The implantable medical device of claim 6, wherein the flared region terminates in a rolled flange.

9. The implantable medical device of claim 8, wherein the rolled flange terminates in a plurality of anti-migration features.

10. The implantable medical device of claim 9, wherein the plurality of anti-migration features is a plurality of teeth, wherein the plurality of teeth is configured to contact a tissue wall when the retention member is in a first configuration and remain in contact with the tissue wall when the retention member is in a second configuration.

11. The implantable medical device of claim 10, wherein the plurality of anti-migration features is uncoated, and wherein at least the saddle region is coated.

12. The implantable medical device of claim 1, wherein the elongated body, the saddle region, and the retention member are formed of interwoven filaments.

13. The implantable medical device of claim 1, wherein the retention member includes a first retention member at the first end and a second retention member at the second end.

14. The implantable medical device of claim 13, wherein the first retention member and the second retention member are the same shape and same size.

15. The implantable medical device of claim 1, wherein the retention member includes a longitudinal projection extending therefrom, and wherein the longitudinal projection is:
configured to be disposed in a gap between a tissue wall and the retention member when the retention member is in a first configuration; and
configured to contact the tissue wall when the retention member is in a second configuration.

16. A self-expanding implantable medical device comprising:
an elongated body having a first end, a second end, and a lumen extending between the first end and the second end;
retention members including a first retention member at the first end and a second retention member at the second end, wherein the retention members extend substantially traverse to the longitudinal axis of the implantable medical device and are configured to undergo elastic deformation between a first configuration in an absence of an application of a force along a longitudinal axis of the implantable medical device and a second configuration responsive of the application of the force; and
a longitudinally expandable saddle region defined between the first retention member and the second retention member, wherein the longitudinally expandable saddle region is configured to extend between a first tissue wall and a second tissue wall, the first retention member is configured to anchor the implantable medical device with respect to the first tissue wall, the second retention member is configured to anchor the implantable medical device with respect to the second tissue wall.

17. The self-expanding implantable medical device of claim 16, wherein the longitudinally expandable saddle region has:
a first length along the longitudinal axis of the longitudinally expandable implantable medical device in the absence of the application of the force;
a second length along the longitudinal axis responsive to the application of the force, wherein the second length is larger than the first length, and
a difference between the second length and the first length is in a range from about 5 millimeters to about 2 centimeters.

18. A self-expanding implantable stent comprising:
an elongated body having a first end, a second end, and a lumen extending between the first end and the second end;
rolled retention members including a first rolled retention member at the first end and a second rolled retention member at the second end, wherein the first and second rolled retention members extend substantially traverse to the longitudinal axis of the implantable medical device and are configured to undergo elastic deformation between a first configuration in an absence of an application of a force along a longitudinal axis of the implantable medical device and a second configuration responsive of the application of the force;
a saddle region defined between the first rolled retention member and the second rolled retention member, wherein the saddle region is configured to extend between a first tissue wall and a second tissue wall;
a first plurality of anti-migration features extending substantially longitudinally from an end of the first rolled retention member and being configured to anchor the implantable medical device with respect to the first tissue wall; and
a second plurality of anti-migration features extending substantially longitudinally from an end of the second rolled retention member and being configured to anchor the implantable medical device with respect to the second tissue wall.

19. The self-expanding implantable stent of the claim 18, wherein:
the rolled retention members have a substantially circular cross-sections;
the first plurality of anti-migration features comprises teeth that are disposed uniformly about a substantially circular end of the first rolled retention member; and
the second plurality of anti-migration features comprises teeth that are disposed uniformly about a substantially circular end of the second rolled retention member.

20. The self-expanding implantable stent of claim 18, wherein:
the first rolled retention member is configured to unroll in a first direction about the longitudinal axis of the implantable medical device responsive to the application of the force; and
the second rolled retention member is configured to unroll in a second direction that is opposite from the first direction.
